(19) United States
(12) Patent Application Publication
(10) Pub. No.: US 2023/0355118 A1
(43) Pub. Date: Nov. 9, 2023

(54) SYSTEM AND METHOD FOR MONITORING PHYSIOLOGICAL PARAMETERS BASED ON CEREBROSPINAL FLUID PRESSURES TAKEN AT TWO OR MORE LOCATIONS

(71) Applicant: EnClear Therapies, Inc., Newburyport, MA (US)

(72) Inventors: Rajan Patel, Somerville, MA (US); Gregory Martin, Boston, MA (US); Mark Gurguis, Newburyport, MA (US); Raiden Skodowski, Newburyport, MA (US)

(21) Appl. No.: 18/132,174

(22) Filed: Apr. 7, 2023

Related U.S. Application Data
(60) Provisional application No. 63/328,579, filed on Apr. 7, 2022.

(51) Int. Cl. A61B 5/024; A61B 5/00; A61B 5/02; A61B 5/08; A61B 5/03

(52) U.S. Cl. CPC A61B 5/024; A61B 5/7257; A61B 5/02007; A61B 5/7246; A61B 5/0816; A61B 5/031

(57) ABSTRACT

Monitoring cardiopulmonary function of a patient based on cerebrospinal fluid (CSF) pressures involves fluidly communicating a first catheter with a first CSF access site of the patient; fluidly communicating a second catheter with a second CSF access site of the patient; providing, using a first pressure sensor in fluid communication with the first catheter, first pressure readings associated with the first CSF access site; providing, using a second pressure sensor in fluid communication with the second catheter, second pressure readings associated with the second CSF access site; and determining a cardiopulmonary function parameter associated with the patient based on first and second pressure signals derived from the first and second pressure readings. The first CSF access site may be associated with a brain ventricle of the patient and the second CSF access site may be associated with the patient's lumbar.

### RELATED APPLICATIONS

This patent application claims the benefit of U.S. Provisional Patent Application No. 63/328,579 entitled SYSTEM AND METHOD FOR MANAGING CSF FLOWS filed Apr. 7, 2022, which is hereby incorporated herein by reference in its entirety.

This patent application is related to the following patent applications filed on Sep. 29, 2021 with the same applicant and some common inventors. All of these patent applications are incorporated herein, in their entireties, by reference.
U.S. application Ser. No. 17/489,620 published as U.S. Published Patent Application No. US 2022-0096743 (Attorney Docket 120902-10804),
U.S. application Ser. No. 17/062,440 issued as U.S. Pat. No. 11,278,657 (Attorney Docket 120902-10103)
U.S. application Ser. No. 17/153,548 issued as U.S. Pat. No. 11,419,921 (Attorney Docket 120902-10203)
U.S. application Ser. No. 17/489,625 published as U.S. Published Patent Application No. US 2022-0096744 (Attorney Docket 120902-10805)
U.S. application Ser. No. 17/495,682 published as U.S. Published Patent Application No. US 2022-0105322 (Attorney Docket 120902-10903) and
U.S. application Ser. No. 17/489,633 published as U.S. Published Patent Application No. US 2022-0096745 (Attorney Docket 120902-10806).

### GOVERNMENT RIGHTS

None

### FIELD

Illustrative embodiments generally relate to medical devices and methods and, more particularly, illustrative embodiments relate to devices and methods for managing subarachnoid fluid, such as cerebrospinal fluid (“CSF”), and/or drug delivery that may be used to treat neurodegenerative disorders.

### BACKGROUND

Intrathecal drug delivery via the cerebrospinal fluid presents a number of safety issues. In particular, it can present significant risk to a patient who reacts negatively with an abnormal heart rate or respiration rate during such a procedure.

### SUMMARY OF VARIOUS EMBODIMENTS

In accordance with certain embodiments, monitoring cardiopulmonary function of a patient based on cerebrospinal fluid (CSF) pressures involves receiving, from a first pressure sensor in fluid communication with a first catheter, first pressure readings associated with a first CSF access site; receiving, from a second pressure sensor in fluid communication with a second catheter, second pressure readings associated with a second CSF access site; and determining a cardiopulmonary function parameter associated with the patient based on first and second pressure signals derived from the first and second pressure readings.

In various alternative embodiments, the determining may involve applying a Fourier Transform to the first and second pressure signals. The determining may involve forming a first Fourier Transform from the first pressure signals, forming a second Fourier Transform from the second pressure signals, and locating at least one peak from the first and second Fourier Transforms, the at least one peak corresponding with the cardiopulmonary function parameter. The determining may involve locating a point of intersection between the first and second pressure signals, the point of intersection corresponding with the cardiopulmonary function parameter. The determining may involve applying a sine wave to one or both of the first and second pressure signals, wherein the cardiopulmonary function parameter is based on one or more of the amplitude, offset, frequency, slope, and period of the sine wave. The determining may involve determining the slope of the first and/or second pressure signals, the cardiopulmonary function parameter being a function of the slope. The determining may involve applying two or more of a Fourier Transform, sine wave, or slope techniques to the first and second pressure signals and to use the result of that application to determine the cardiopulmonary function parameter. The determining may involve applying a peak/valley process to the first and second pressure signals to determine the cardiopulmonary function parameter.

Alternative embodiments may involve monitoring the cardiopulmonary function parameter and producing indicia when the cardiopulmonary function parameter extends beyond a prescribed range, the indicia being visual, audio, and/or tactile indicia.

Alternative alternative embodiments may involve taking a baseline reading using the first and/or second pressure signals to determine a baseline for the cardiopulmonary function parameter, and storing the baseline reading in a database.

In various alternative embodiments, the first CSF access site may be associated with a brain ventricle of the patient and the second CSF access site may be associated with the patient's lumbar. A catheter may have a first end coupled with the first CSF access site and a second end coupled with the second CSF access site, the first pressure sensor being closer to the first end than the second end, and the second pressure sensor being closer to the second end than the first end. The cardiopulmonary function parameter may include at least one of heart rate, respiration rate, heart rate amplitude, respiration rate amplitude, or vessel compliance.

Alternative embodiments may involve receiving, from a third pressure sensor in fluid communication with a third catheter, third pressure readings associated with a third CSF access site, wherein determining comprises determining the cardiopulmonary function parameter associated with the patient based on third pressure signals derived from the third pressure readings in combination with the first and second pressure signals.

Alternative embodiments may involve characterizing a first pressure contribution associated with the first catheter; producing the first pressure signals based on the first pressure readings and the first pressure contribution; characterizing a second pressure contribution associated with the second catheter; and producing the second pressure signals based on the second pressure readings and the second pressure contribution.

In accordance with another embodiment of the invention, a method of determining respiration rate or heart rate fluidly communicates a catheter with a patient's brain ventricle and the patient's lumbar. Next, the method uses a ventricle pressure sensor, in fluid communication with the catheter, to detect the brain ventricle pressure, consequently producing a ventricle pressure signal. In a corresponding manner, the method uses a lumbar pressure sensor, in fluid communication with the catheter, to detect the lumbar pressure, consequently producing a lumbar pressure signal. The method uses the ventricle pressure signal and the lumbar pressure signal to determine respiration rate and/or heart rate.

The method may use the signals in a number of manners to determine the relevant patient physiological parameters. Among other things, the method may:
apply a Fourier Transform to the lumbar and ventricle pressure signals,
form a Fourier Transform from the lumbar and ventricle pressure signals and locate at least one peak from the Fourier Transform. The at least one peak corresponds with at least one of the respiration rate and the heart rate.
locate a point of intersection between the lumbar and ventricle pressure signals, where the point of intersection corresponds with at least one of the heart rate and the respiration rate.
apply a sine wave to one or both to the lumbar and ventricle pressure signals. In this case, the method may ascertain the one or more physiological parameters based on one or more of the amplitude, offset, frequency, slope, and period of the sine wave. Among other things, the one or more physiological parameters may include at least one of the heart rate, respiration rate, cerebral perfusion pressure, intracranial pressure, heart rate amplitude, respiration rate amplitude, vessel elasticity/impedance, cerebral compliance, and CSF production rate.
determine the slope of the lumbar and/or ventricle pressure signals, the heart rate and/or the respiration rate being a function of the slope.
apply two or more of a Fourier Transform, sine wave, and slope techniques to the ventricle and lumbar pressure signals and use the result of that application to determine the respiration rate and/or the heart rate

Some embodiments monitor the heart rate and/or the respiration rate and produce indicia when the heart rate and/or the respiration rate extend beyond a prescribed range relative to a baseline reading. Among other things, the indicia may include visual, audio, and/or tactile indicia. Alternatively or in addition, the method may take a baseline reading using the lumbar and/or ventricle pressure signals to determine heart rate and/or respiration rate. The baseline reading may be stored in a database.

The catheter may be considered to have a ventricle end coupled with the ventricle, and a lumbar end coupled with the lumbar. The lumbar pressure sensor is closer to the lumbar end than the ventricle end and, in a corresponding manner, the ventricle pressure sensor is closer to the ventricle end than the lumbar end.

In accordance with another embodiment, a CSF system has a catheter with a lumbar end and a ventricle end. The system also has a lumbar pressure sensor in fluid communication with the lumbar end so that the lumbar pressure sensor is closer to the lumbar end than to the ventricle end. As with other embodiments, the lumbar pressure sensor is configured to produce a lumbar pressure signal. The system also has a ventricle pressure sensor in fluid communication with the lumbar end and as such, closer to the ventricle end than to the lumbar end. The ventricle pressure sensor is configured to produce a ventricle pressure signal. Importantly, the system also has a physiological parameter monitor operatively coupled with the ventricle pressure sensor and the lumbar pressure sensor. The physiological parameter monitor is configured to use the ventricle and lumbar pressure signals to determine the respiration rate and/or the heart rate.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those skilled in the art should more fully appreciate advantages of various embodiments of the invention from the following “Description of Illustrative Embodiments,” discussed with reference to the drawings summarized immediately below.

FIG. 1 schematically shows a cerebrospinal fluid circuit that may be used with illustrative embodiments of the invention.

FIG. 2 schematically shows an external catheter configured in accordance with illustrative embodiments.

FIG. 3A shows a high level surgical flow process in accordance with illustrative embodiments.

FIGS. 3B and 3C schematically show bidirectional pump circuits that enable flow in two opposite directions (FIG. 3C between right and left ventricles in the brain) in accordance with illustrative embodiments.

FIG. 4 schematically shows a system interface in accordance with illustrative embodiments.

FIG. 5 schematically shows a sensor element and load cell interface coupled to CSF circulation tubing in accordance with illustrative embodiments.

FIG. 6 schematically shows a flow with variables, parameters, and outputs in accordance with illustrative embodiments.

FIG. 7 shows a process of managing fluid flow in accordance with illustrative embodiments.

FIG. 8A shows a process of determining parameters with pressure signals using a Fourier Transform technique in accordance with illustrative embodiments.

FIGS. 8B and 8C graphically show examples of the process of FIG. 8A.

FIG. 9A shows a process of determining parameters with pressure signals using a Sine Wave technique in accordance with illustrative embodiments.

FIG. 9B graphically show an example of the process of FIG. 9A.

FIG. 10A shows a process of determining parameters with pressure signals using a linear regression/slope technique in accordance with illustrative embodiments.

FIG. 10B graphically show an example of the process of FIG. 10A.

FIG. 11A shows a peak/valley process of determining heart and respiration parameters in accordance with illustrative embodiments.

FIG. 11B graphically shows an example of the process of FIG. 11A.

### DESCRIPTION OF ILLUSTRATIVE EMBODIMENTS

Illustrative embodiments use pressures detected from at least two cerebrospinal fluid (CSF) access locations (e.g., a brain ventricle and the lumbar) to determine various physiological values. For example, a fluid circuit fluidly connecting two or more CSF access regions can include pressure sensors in appropriate locations to detect those pressures. One or more of a plurality of different techniques then convert those pressures into the desired physiological values. Among others, those physiological values may include one or more cardiopulmonary function parameters (e.g., heart rate frequency, heart rate amplitude, respiration rate frequency, respiration rate amplitude, and/or vessel compliance), which may then be used, e.g., to manage drug delivery and/or improve patient safety protocols. Details of illustrative embodiments are discussed below. It should be noted that, while certain exemplary embodiments are described below with reference to pressure measurements taken at a patient's brain ventricle and lumbar, the described techniques can apply more generally to other CSF access locations such as, for example, two brain ventricles, two brain ventricles plus lumbar, brain ventricle plus lumbar plus cisterna magna, etc.

Many neurodegenerative diseases have been tied to the accumulation of biomolecules (e.g., toxic proteins) contained in cerebrospinal fluid (CSF) or other fluids (e.g., interstitial fluid) within the subarachnoid space (SAS) of a mammalian subject. Problematically, these (e.g., toxic) biomolecules may be secreted and then transported by the CSF to other cells in the body, and that process may occur over the span of years. For example, dipeptide repeat proteins (DPRs) and/or TDP-43 have been implicated in neuronal death in the pathology of amyotrophic lateral sclerosis (ALS, or Lou Gehrig's disease), Alzheimer disease (AD), frontotemporal degeneration (FTD), Parkinson's disease (PD), Huntington's disease (HD), and progressive supranuclear palsy (PSP), to name just a few. Hence, research has focused primarily on the removal of harmful DPRs. Techniques for removing DPRs and/or TDP-43 have included: shunting CSF from the CSF space, diluting the CSF (e.g., with an artificial fluid), administering a drug into the CSF, conditioning the CSF, and/or manipulating CSF flow.

Recent breakthrough techniques for handling this problem include ameliorating the CSF, and treating a neurological disorder by removing or degrading a specific (toxic) protein.

Amelioration, as used in various embodiments, involves systems and methods for ameliorating a fluid in the subarachnoid space (SAS) (e.g., a cerebrospinal fluid (CSF), an interstitial fluid (ISF), blood, and the like) of a mammalian subject, unless otherwise particularly distinguished (e.g., referred to as solely CSF). Representative systems may be completely or partially implanted within the body of the mammalian subject (discussed below). Within the body, the systems and/or components thereof may also be completely or partially implanted within the SAS and exposed to the exterior via a port 16 (e.g., a medical valve that provides selective access to the interior system components). These systems execute processes that may occur entirely in-vivo, or some steps that occur extracorporeally. Illustrative embodiments ameliorate with a CSF circuit, discussed below.

Amelioration, for the purpose of illustration, may include changing the physical parameters of the fluid, as well as digestion, removal, immobilization, reduction, and/or alteration, to become more acceptable and/or inactivation of certain entities, including: target molecules, proteins, agglomerations, viruses, bacteria, cells, couples, enzymes, antibodies, substances, and/or any combination thereof. For example, in some embodiments and applications, amelioration may refer to removing toxic proteins from or conditioning one or more of the blood, interstitial fluid, or glymph contained therein, or other fluid, as well as the impact that this removal has on treating diseases or conditions that affect various bodily functions, (i.e., improving the clinical condition of the patient). Moreover, amelioration may be performed by any one of: digestion, enzymatic digestion, filtration, size filtration, tangential flow filtering, countercurrent cascade ultrafiltration, centrifugation, separation, magnetic separation (including with nanoparticles and the like), electrophysical separation (performed by means of one or more of enzymes, antibodies, nanobodies, molecular imprinted polymers, ligand-receptor complexes, and other charge and/or bioaffinity interactions), photonic methods (including fluorescence-activated cell sorting (FACS), ultraviolet (UV) sterilization, and/or optical tweezers), photo-acoustical interactions, chemical treatments, thermal methods, and combinations thereof. Advantageously, various embodiments or implementations of the present invention may reduce levels of toxicity and, after reduced, facilitate maintaining the reduced levels over time.

The extent of amelioration, as reflected by the concentration of the target biomolecules, may be detected through a variety of means. These include optical techniques (e.g., Raman, coherent Stokes, and anti-Stokes Raman spectroscopy; surface enhanced Raman spectroscopy; diamond nitrogen vacancy magnetometry; fluorescence correlation spectroscopy; dynamic light scattering; and the like) and use of nanostructures such as carbon nanotubes, enzyme linked immunosorbent assays, surface plasmon resonance, liquid chromatography, mass spectrometry, circular proximity ligation assays, and the like.

Amelioration may include the use of a treatment system (e.g., UV radiation, IR radiation), as well as a substance, whose properties make it suitable for amelioration. Amelioration of CSF or ameliorated CSF—which terms may be used interchangeably herein—refers to a treated volume of CSF in which one or more target compounds have been partially, mostly, or entirely removed. It will be appreciated that the term “removed,” as used herein, can refer not only to spatially separating, as in taking away, but also effectively removing by sequestering, immobilizing, or transforming the molecule (e.g., by shape change, denaturing, digestion, isomerization, or post-translational modification) to make it less toxic, non-toxic or irrelevant.

The term, “ameliorating agent” generally refers to a material or process capable of ameliorating a fluid, including enzymes, antibodies, or antibody fragments, nucleic acids, receptors, anti-bacterial, anti-viral, anti-DNA/RNA, protein/amino acid, carbohydrate, enzymes, isomerases, compounds with high-low biospecific binding affinity, aptamers, exosomes, ultraviolet light, temperature change, electric field, molecular imprinted polymers, living cells, and the like. Additional details of amelioration are taught by the incorporated related applications, as well as in PCT Application No. PCT/US20/27683, filed on Apr. 10, 2020, the disclosure of which is incorporated herein, in its entirety, by reference. In a similar manner, details for further treatments are taught by PCT Application No. PCT/US19/042880, filed Jul. 22, 2019, the disclosure of which is incorporated herein, in its entirety, by reference.

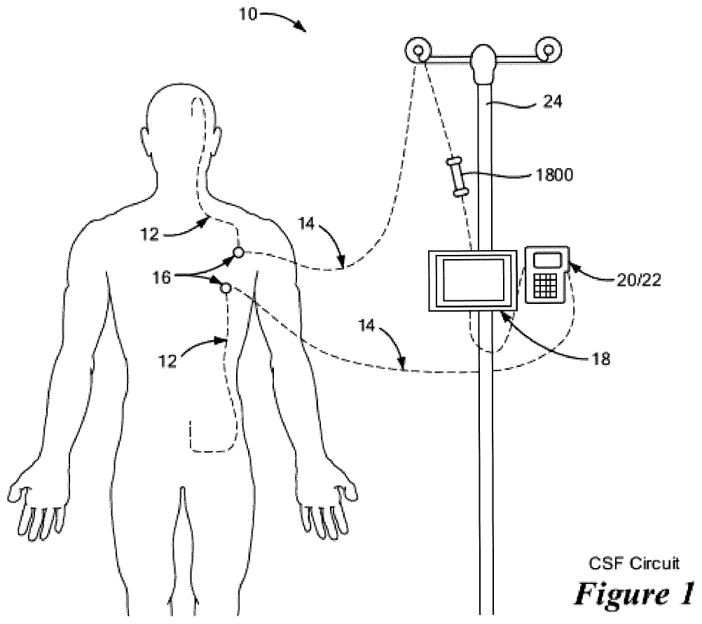

To control CSF flow within the body (e.g., through the ventricle), illustrative embodiments form a CSF circuit/channel (identified by reference number “10”) that manages fluid flow in a closed loop. FIG. 1, for example, shows one embodiment of such a CSF circuit 10. In this example, internal catheters 12 (also referred to generically as “tubing” or the like) positioned in-vivo/interior to the body fluidly couple together via the subarachnoid space. To that end, a first internal catheter 12 fluidly couples a prescribed region of the brain (e.g., the ventricle) to a first port 16, which itself is configured and positioned to be accessible by external components. In a corresponding manner, a second catheter couples the lumbar region or the lower abdomen of the subarachnoid space with a second port 16 that, like the first port 16, also is configured to be positioned and accessible by external components.

The first and second ports 16 may be those conventionally used for such purposes, such as a valved Luer-lock or removable needle. The first and second internal catheters 12 thus may be considered to form a fluid channel extending from the first port 16, to the ventricle, down the spine/subarachnoid space to the lumbar, and then to the second port 16. These internal components, which may be referred to as “internal CSF circuit components,” are typically surgically implanted by skilled professionals in a hospital setting.

The CSF circuit 10 also has external components (referred to as “external CSF circuit components). To that end, the external CSF circuit components include at least two fluid conduits 14. Specifically, the external CSF circuit components include a first external fluid conduit 14, that couples with the first port 16 for access to the ventricle. The other end of the first external conduit 14 is coupled with a management system, which includes one or more CSF pumps (all pumps are generically identified in the figures as reference number “18”), one or more user interface/displays 20, one or more drug pumps 18, and a control system/controller 22. The fluid external fluid conduit 14 may be implemented as a catheter and thus, that term may be used interchangeably with the term “conduit” and be identified by the same reference number 14.

Illustratively, this management system is supported by a conventional support structure (e.g., a hospital pole 24 in FIG. 1). To close the CSF circuit 10, a second external catheter 14 extends from that same CSF management system and couples with the second port 16 and the management system. This management system and external catheters 14 therefore form the exterior part of a closed CSF circuit 10 for circulating the CSF and therapeutic material.

It should be noted that the CSF circuit 10 may have one or more components between the first and second ports 16 and the respective removable connections of the first and second external catheters 14. For example, the first port 16 may have an adapter that couples with the first external catheter 14, or another catheter with a flow sensor may couple between such external catheter 14 and port 16. As such, this still may be considered a removable connection, albeit an indirect fluid connection. There may be corresponding arrangements with the other end of the first external catheter 14, as well as corresponding ends of the second external catheter 14. Accordingly, the connection can be a direct connection or an indirect connection.

The first and second external catheters 12 and 14 preferably are configured to have removable connections/couplings with the management system, as well as their respective ports 16. Examples of removable couplings may include a screw-on fit, an interference fit, a snap-fit, or other known removable couplings known in the art. Accordingly, a removable coupling or removable connection does not necessarily require that one forcibly break, cut, or otherwise permanently break the ports 16 for such a connection or disconnection. Some embodiments, however, may enable a disconnection form the first and/or second ports 16 via breaking or otherwise, but the first and/or second ports 16 should remain in-tact to receive another external catheter 14 (e.g., at the end of life of the removed external catheter 14).

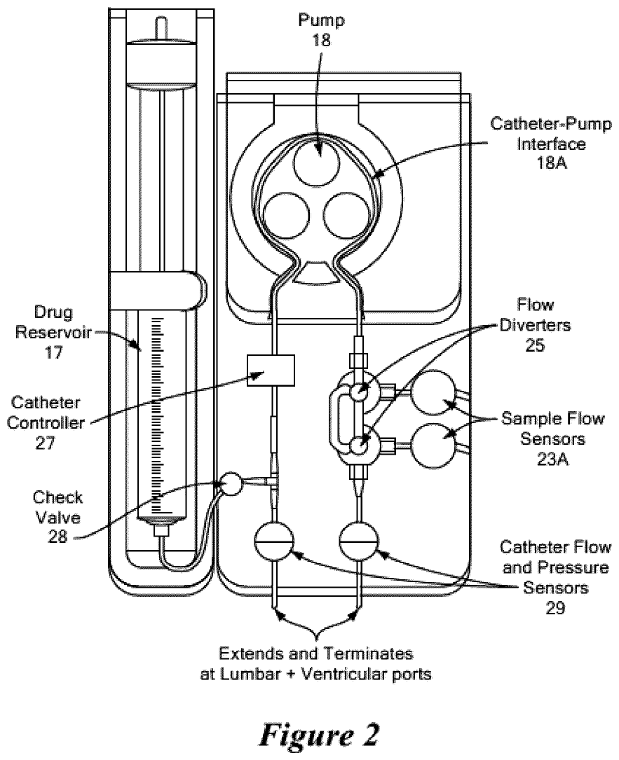

FIG. 2 schematically shows more details of the first and/or second external conduits/catheters 14. This figure shows an example of an external catheter 14 operating with other parts of the system. As shown, in this example, the system receives an optional drug reservoir 17 (e.g., a single-use syringe) configured to deliver a dose of therapeutic material (e.g., a drug) that fluidly couples with the catheter 14 via a check valve 28 and T-port on the catheter 14. In addition, the catheter 14 is coupled with a mechanical pump 18 and also preferably includes a sample port 23 with flow diverters 25 for diverting flow toward or away from a sample port 23. The sample port 23 preferably has sample port flow sensors 23A to track samples.

Some embodiments may be implemented as a simple catheter having a body forming a fluid-flow bore with removably couplable ends (or only one removably couplable end). Illustrative embodiments, however, add intelligence to make one or both of these external catheters 14 “smart” catheters, effectively creating a more intelligent flow system. For example, either one or both of the external catheters 14 can have a processor, ASIC, memory, EEPROM (discussed below), FPGAs, RFID, NFC, or other logic (generally identified as reference number “27”) configured to collect, manage, control the device, and store information for the purposes of security, patient monitoring, catheter usage, or communicating with the management system 19 to actively control fluid dynamics of the CSF circuit 10. Among other things, the management system 19 may be configured to coordinate with an EEPROM 27 to control CSF fluid flow as a function of the therapeutic material infusion flow added to the CSF circuit 10 (discussed below) via the check valve 28 at the output of the drug reservoir 17.

As shown in FIG. 2, one embodiment of the external catheter 14 has electrically erasable programmable read-only memory, EEPROM 27, (or other logic/electronics) that can be implemented to accomplish a variety of functions. Among others, the EEPROM 27 can ensure that the CSF circuit 10 and its operation is customized/individualized to a patient, a treatment type, a specific disease, and/or a therapeutic material. For example, in response to reading information stored in the EEPROM 27, the control system 22 may be configured to control fluid flow as a function of the therapeutic material.

Importantly, as a disposable device, the EEPROM 27 or other logic of the external catheter 14 can be configured to provide alerts, and/or produce or cause production of some indicia (e.g., a message, visual indication, audio indication, etc.) indicating that the external catheter 14 has reached an end of its lifecycle, or indicating how much of its lifecycle remains. For example, an external surface of the catheter 14 may have a tag that turns red when the EEPROM 27 and/or other logic 27 determines that the external catheter 14 has reached its full lifetime use. For example, the external catheter 14 may be considered to have a usage meter, implemented as some logic or EEPROM 27, configured to track use of the CSF fluid conduit 14 to help ensure it is not used beyond its rated lifetime. Moreover, the logic or EEPROM 27 can register with the control system 22 to start use timers to reduce tampering or use beyond a lifetime.

Some embodiments have a printable circuit board (PCB) equipped with a wireless interface (e.g., Bluetooth antenna) or a hardware connection configured to communicate the pump 18 and/or control system 22. The external catheter 14 can be configured to time out after a certain period, capture data, and communicate back and forth with the control system 22 or other off-catheter or on-catheter apparatus to share system specifications and parameters. The intelligent flow catheter 14 can be designed with proprietary connections such that design of knockoffs or cartridges 26 (discussed below) can be prevented to ensure safety and efficacy of the CSF circuit 10 and accompanying processes.

In addition to the management logic, the external catheter(s) 14 also may have a set of one or more flow sensors and/or a set of one or more pressure sensors. Both of those sensors are shown generically at reference number 29, and may be located upstream or downstream from their locations in FIG. 2. For example, the left sensor(s) 29 generically shown in FIG. 2 can be a flow sensor, pressure, or both a flow sensor and pressure. The same can be said for the other sensor(s) 29 generically shown in FIG. 2. They preferably are positioned between the ports 16 on the body and the remaining components as shown.

Of course, the flow sensor(s) 29 may be configured to detect flow through the bore of the catheter body, while the pressure sensor(s) 29 may be configured to detect pressure within the bore of the body. In preferred embodiments, the pressure sensors are configured to detect pressure in the lumbar region and pressure in the ventricle region of the brain. Among other functions, the flow sensor(s) 29 may monitor flow rate of fluid through the conduit bore and/or total flow volume through the conduit bore.

The catheter 14 preferably is configured to have different hardness values at different locations. Specifically, illustrative embodiments may use a mechanical pump 18, as shown and noted above. The pump 18 may periodically urge a compressive force along that portion of the catheter 14 it contacts at its interface 18A with the catheter 14. The outlet of the pump 18 in this case may be the portion of the catheter 14 that is receiving the output of a neighboring compressed catheter portion (e.g., a portion that is adjacent to the compressed catheter portion(s). To operate efficiently, illustrative embodiments form the catheter 14 to have a specially configured hardness at that location (e.g., 25-35 Shore A). Diameter also is important for flow and thus, one skilled in the art should determine appropriate diameters as a function of performance and durometer/hardness. Preferably, the catheter portion that contacts the pump 18 is softer than the remainder of the catheter 14, although both could have the same hardness. Accordingly, the catheter preferably has a variable hardness along its length and may even have a variable diameter.

Alternative embodiments may provide an open-loop CSF fluid circuit 10. For example, the CSF fluid circuit 10 may have an open bath (not shown) to which fluid is added and then removed. The inventors expect the closed-loop embodiment to deliver better results, however, than those of the open-loop CSF fluid circuit 10.

Illustrative embodiments are distributed to healthcare facilities and/or hospitals as one or more kits. For example, one more inclusive kit may include the internal and external catheters 12 and 14. Another exemplary kit may include just the internal catheters 12 and the ports 16 (e.g., for a hospital), while a second kit may have the external catheters 14 and/or a single-use syringe. Other exemplary kits may include the external catheters 14 and other components, such as the management system 19, pressure sensors, and/or a CSF treatment cartridge 1800.

Accordingly, when coupled, these pumps 18, valves (generally identified by reference number 28), internal and external catheters 14, and other components may be considered to form a fluid conduit/channel that directs CSF to the desired locations in the body in a prescribed or controlled manner. It should be noted that although specific locations and CSF containing compartments are discussed, those skilled in the art should recognize that other compartments can be managed (e.g., the lateral ventricles, the lumbar thecal sac, the third ventricle, the fourth ventricle, and/or the cisterna magna). Rather than accessing the ventricle and the lumbar thecal sac, both lateral ventricles could be accessed with the kit. With both internal catheters 12 implanted, CSF may be circulated between the two lateral ventricles, or a drug could be delivered to both ventricles simultaneously. Pressure readings may be made on a continuous or intermittent basis at any of these desired locations.

Pressure sensors 29 may be positioned in other locations in the CSF circuit. Accordingly, discussion in a specific location is by example only and not intended to limit some other embodiments. Indeed, various embodiments require a precise placement of the pressure sensors to effectively collect the requisite pressure data.

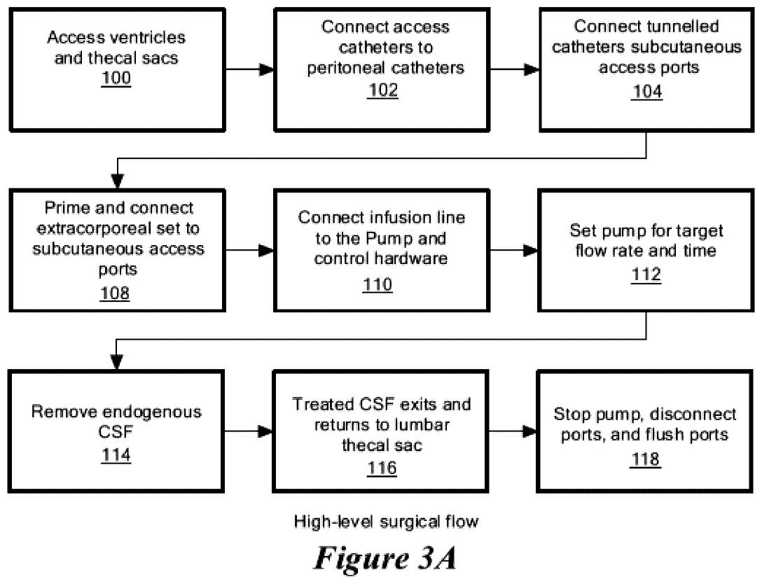

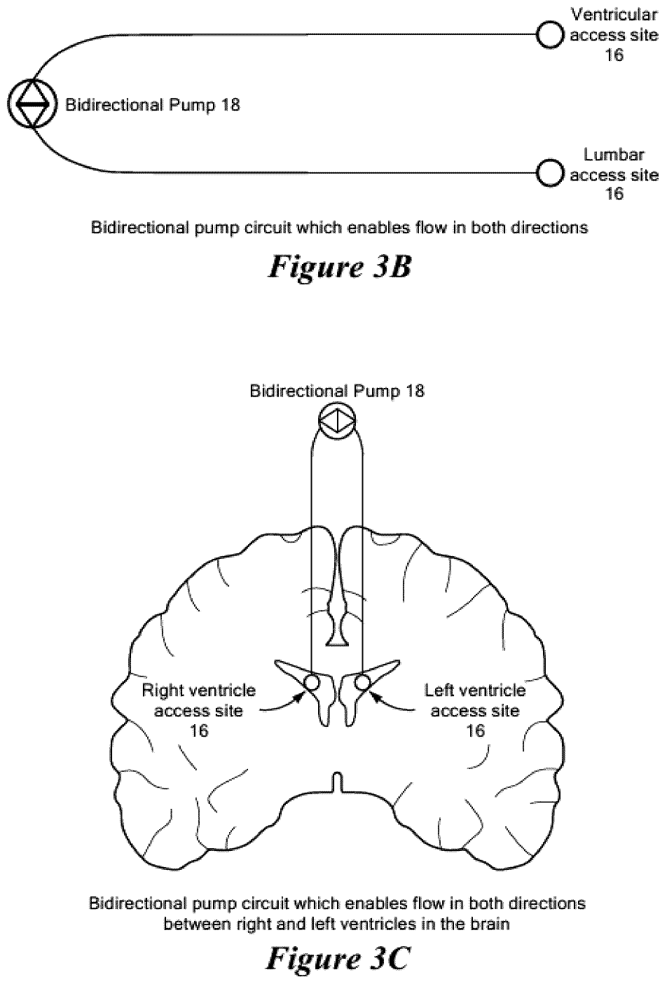

FIG. 3A shows a high level surgical flow process that may incorporate the CSF circuit 10 of FIG. 1 in accordance with illustrative embodiments of the invention. It should be noted that this process is substantially simplified from a longer process that normally would be used to complete the surgical flow. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 100 by setting up the internal catheters 12 inside the patient. To that end, step 100 accesses the ventricles and thecal sacs using standard catheters and techniques, thus providing access to the CSF. Step 102 then connects access catheters 12 to peritoneal catheters 12, which are tunneled subcutaneously to the lower abdomen. The tunneled catheters 12 then are connected at step 104 to the ports 16 implanted in the abdomen.

At this point, the process sets up an extracorporeal circulation set (i.e., the external catheters 14, or the “smart catheters” in some embodiments). To that end, step 106 may prime and connect the extracorporeal circulation set 14 to the subcutaneous access ports 16. Preferably, this step uses an extracorporeal circulation set, such as one provided by Enclear Therapies, Inc. of Newburyport, MA, and/or the external catheters 14 discussed above. The process continues to step 110, which connects an infusion line or other external catheter 14 to the management system 19, and then sets the target flow rate and time. At this point, setup is complete and treatment may begin (step 112).

The process then removes endogenous CSF from the ventricle. This CSF may then be passed through a digestion region (e.g., through a cartridge 1800 or other structure having a specific digesting material), where certain target proteins in the CSF are digested. For example, the cartridge 1800 may have an inner plenum space filled with a plurality of (e.g., porous, chromatography resin) beads that have been compression packed. To prevent constituents from entering or escaping from the cartridge 1800, a filter membrane may be disposed at the first end of the cartridge 1800 and a second filter membrane may be disposed at the second end of the cartridge 1800. In some applications, the ameliorating agent may be decorated on the beads.

In some applications, the cartridge 1800 may be compression packed with a chromatography resin (e.g., agarose, epoxy methacrylate, amino resin, and the like) that has a protease covalently bonded (i.e. immobilized) to the three-dimensional resin matrix. The selected protease may be configured to degrade and/or removing target toxic biomolecules by way of proteolytic degradation. The resin may be a porous structure having a particle size commonly ranging between 75-300 micrometers and, depending on the specific grade, a pore size commonly ranging between 300-1800 Å. Thus, at a high level, the cartridge 1800 has ameliorating agent that removes and/or substantially mitigates the presence of toxic proteins from the CSF.

This and similar embodiments may consider this to be an input for the digesting enzyme. Any location providing access to the drug may be considered to be an input for the drug. At step 116, the treated CSF exits the digestion region and is returned via the CSF circuit 10 to the lumbar thecal sac. The process concludes at step 118, which stops the pump 18 when treatment is complete. The management system 19 then may be disconnected and the ports 16 flushed.

Flow direction oscillation and a pulsatile flow pattern could also be produced using a bidirectional pump 18 instead of using valves 28 (e.g., FIG. 3B and FIG. 3C). The pump 18 can be programmed to switch flow directions at a frequency set by the user. While flowing in one direction, the pump 18 can be programmed to pulse by starting and stopping at a frequency also set by the user. Those skilled in the art may use other techniques to provide bidirectional flow.

Various embodiments may set the frequency, flow rate, and other parameters as a function of the requirements and structure of the anatomy, patient specific requirements, and devices used in the treatment (e.g., in the CSF circuit 10). In illustrative embodiments, the actual or calculated intracranial pressure drives the CSF flow rates. For example, as indicators of other physiological values, the pressure signal may indicate a sudden increase in heart rate or steep drop in respiration rate, affecting the CSF circuit operation. Other requirements may include the diameter of the catheters 14 in the CSF circuit 10, physical properties of the drug, the interaction of the drug at the localized region, the properties of the localized region, and other requirements and parameters relevant to the treatment. Those skilled in the art may select appropriate parameters as a function of the requisite properties.

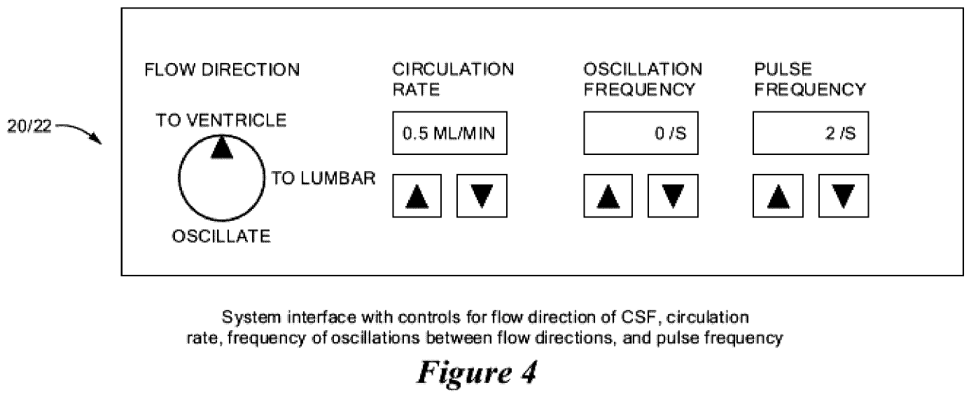

FIG. 4 schematically shows a system interface 20 for control system/controller 22 in accordance with illustrative embodiments. Specifically, whether controlling delivery parameters by pinch valve 28, a bidirectional pump 18, or other means, the delivery profile can be controlled manually with an interface, such as the interface shown in FIG. 4, and/or a delivery profile loaded onto the system. As with the other interfaces, this interface may be a fixed control panel, a graphical user interface on a display device, or a combination of both.

As noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments. Additional details are provided in the above listed patent applications that have been incorporated by reference.

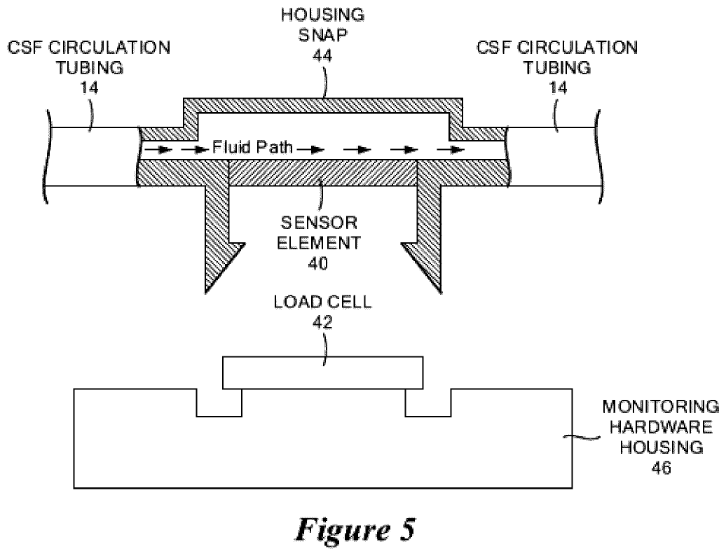

As depicted schematically in FIG. 5, rather than using direct sensors on the patient, the system/circuit 19/10 preferably has monitoring hardware comprising pressure sensors (discussed above but more specifically identified by reference number “42” for this specific pressure sensor, e.g., a load cell) capable of measuring the pressure at the lumbar and the ventricular regions. In one embodiment, these pressure sensors are coupled to a compatible component on the disposable tubing/catheter 14. This compatible component may include a sensor element that is in direct contact with the CSF fluid and capable of communicating with the pressure sensor 42 (e.g., the noted load cell 42) mounted to the monitoring hardware. For example, a sensor element 40 on the tubing/catheter 14 may be in direct communication with the fluid path of a lateral ventricle, as shown in FIG. 5.

Specifically, FIG. 5 illustrates an embodiment of the sensor element 40 and a load cell interface 44 configured to attach to the tubing/catheter 14 to be in direct contact with the CSF fluid. The sensor element 40 can have a housing 46 with a downwardly extending portion to removably couple with the load cell 42 (e.g., a snap-fit). Among other things, the sensor element 40 can include a flexible diaphragm (e.g., a silicone diaphragm) that flexes in response to a pressure stimulus. As the CSF fluid flows through the CSF tubing/catheter 14, the CSF exerts an outward force on the sensor, effectively producing a pressure signal/reading (i.e., producing data representing the pressure in the line).

The monitoring hardware may include the noted housing 46 with processors, memory, etc. having embedded software configured to produce a graphical user interface (“GUI”) on a display. Alternatively or additionally, in some embodiments, the GUI can be implemented as a touch screen. Obtained pressure data is collected, stored in a database (e.g., on the devices shown or on a separate device communicating with the pressure sensor), and can be displayed on the display device where it can be observed by a clinician. The display may show ‘real-time’ data at various sampling frequency, average reading, minimum readings, maximum readings, etc. The system may thus have a physiological parameter monitor configured to use the ventricle and/or lumbar pressure signals to determine the physiological value of interest, such as the respiration rate and/or the heart rate.

As noted above, illustrative embodiments use the ventricular and lumbar pressure sensor data to determine other physiological values (sometimes also referred to as “hidden parameters” or “hidden variables”), such as one or more of the following:
cerebral perfusion pressure,
intracranial pressure or ventricular pressure,
heart rate frequency,
heart rate amplitude,
respiration rate frequency,
respiration rate amplitude,
vessel elasticity/impedance,
cerebral compliance.

To that end, a first catheter is coupled to the patient's brain ventricle at an appropriate ventricular access site and a second catheter is coupled to the patient's lumbar at an appropriate lumbar access site, e.g., as depicted schematically in FIG. 3B. The first and second catheters may be part of a fluid circuit that can include a pump and/or other components, e.g., as depicted schematically in FIG. 3B. The first and second catheters may be part of a fluid circuit, in which case the first and second catheters may be parts of a single catheter (e.g., a catheter having a lumbar end and a ventricle end) or may be separate catheters interconnect by tubing or other fluid channel. For convenience, the fluid circuit including the first and second catheters may be referred to herein collectively as “a catheter.” In any case, pressure sensors (e.g., pressure sensors of the type depicted schematically in FIG. 5 or other pressure sensors) are positioned relative to the first and second catheters for sensing pressures of the brain ventricle and lumbar. It should be noted that embodiments can include more than two catheters and corresponding pressure sensors such that three or more pressure readings can be used to derive the physiological values.

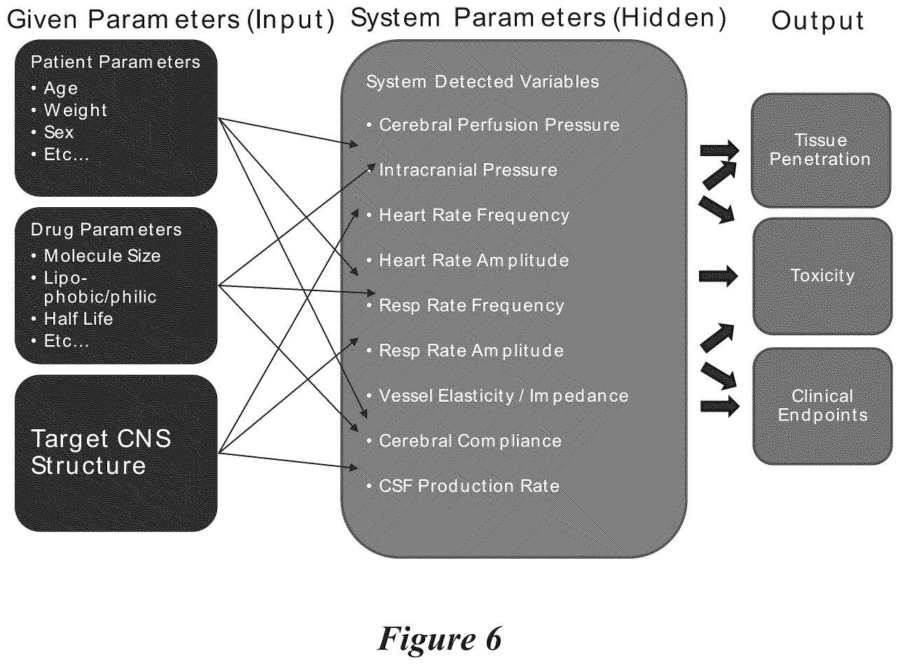

FIG. 6 schematically shows a system that collects data, including pressure data, and uses that data to determine one or more of a plurality of different outcomes. As shown, the system may receive known inputs, such as one or more of:
patient information, such as age, weight, gender, height, health condition, family history, etc.
drug information (if infusing a drug), such as molecular size, lipo-phobic/phillic, half-life, known reactions to certain patient communities, dosage size, concentration, etc.
target central nervous system (“CNS”) structure in the patient's body.

Some embodiments may use a plurality of specific sensors to detect the hidden variables/parameters or, in illustrative embodiments, simply use the pressure sensors to reduce sensor count and cost while often improving system effectiveness. Other embodiments may use a combination of pressure sensors and specific sensors.

As shown in FIG. 6, illustrative embodiments use the given and hidden parameters as input into the system to determine one or more of a variety of important results of the CSF treatment, such as information that is useful with CSF infused drug treatment. These results may relate to the effectiveness and/or safety of a drug treatment, or simply the overall safety of the CSF cycling process. Three examples of such output results include the penetration of a drug into tissue, the current toxicity of the drug, and the clinical endpoints. Other examples may include whether a patient is having a dangerous physical event (e.g., a stroke or heart attack).

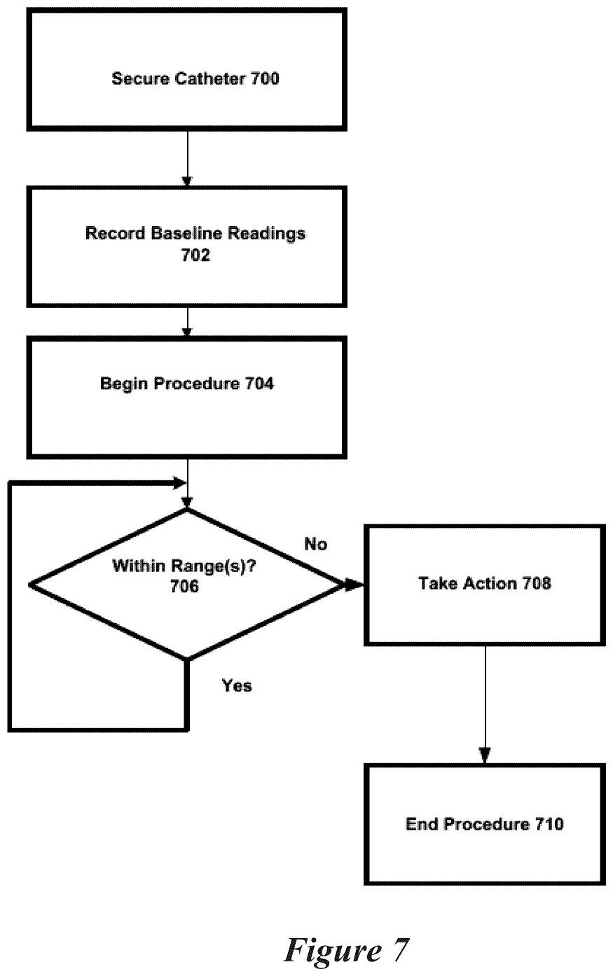

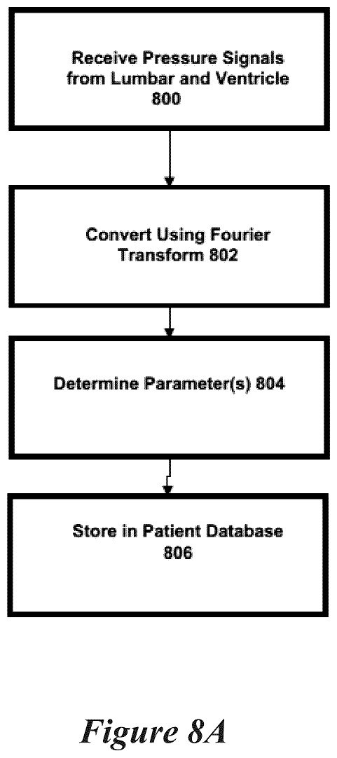

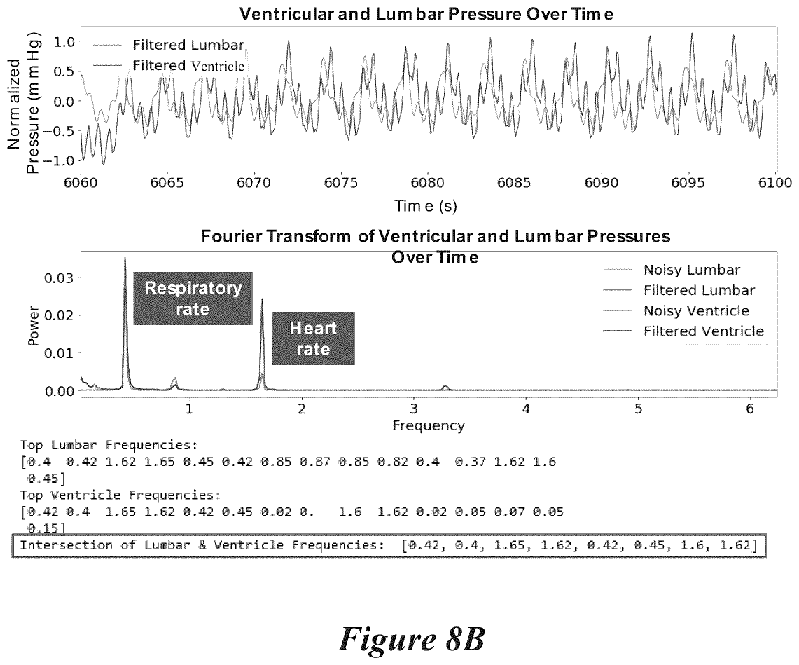

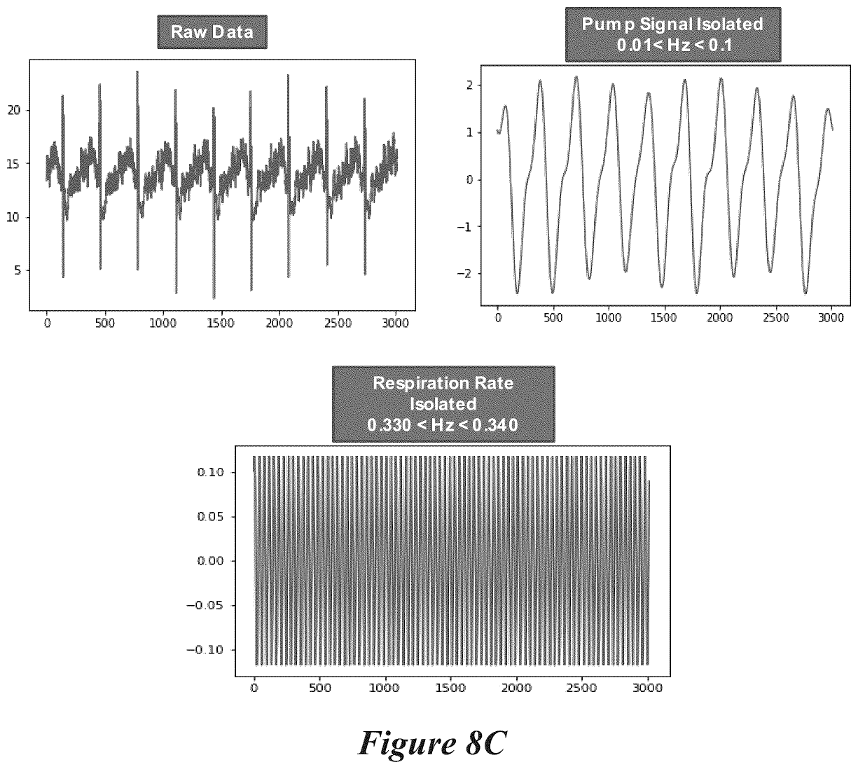

As an example of an application of FIG. 6, FIG. 7 shows a process of managing fluid flow in accordance with illustrative embodiments. As with other figures (e.g., FIG. 3A, discussed above, and FIGS. 8A, 9A, and 10A, discussed below), it should be noted that this process is substantially simplified from a longer process that normally would be used to complete the CSF management process. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 700, which secures the catheter system to the patient. To have a meaningful comparison, patient baseline readings may be obtained and recorded at step 702. Those baseline readings may include any of the noted variable parameters determined from the detected pressure values and/or directly measured variable parameters via specific sensors (e.g., respiratory rate sensor, heart sensor, etc.). Those baseline readings of the variable parameters (or other parameters) may be taken at any of a variety of times, such as hours or days before the current procedure, or at the beginning of the procedure. Preferably, a specialized patient database stored in memory on-system or off-system may store the baseline information in a database for use later in the process or with other processes.

After collecting the baseline readings, the medical procedure may begin (step 704). As noted above, the procedure may include infusing a drug into the patient via their CSF and/or a procedure to ameliorate toxic proteins in the CSF. During this time, the system monitors the variable parameters to ensure that the procedure is proceeding in a desired manner (e.g., in a safe manner). To that end, using the baseline reading and/or other known information relating to the procedure (e.g., from literature or clinical studies), the process tracks certain variable parameters to ensure that they remain within certain ranges during the procedure (step 706). For example, for a specific patient receiving the procedure, the system may set a heart rate range of 50 to 90 beats per minute as an acceptable range. In addition, some embodiments may extend the range to some extent, using the noted baseline and other information, to an “elevated risk” level. For example, if the system detects a heart rate between 45-50 beats per minute or between 90-100 beats per minute, it may require a change in the procedure, such as a reduction or increasing CSF flow rate, changing the infusion rate, etc., or simply emit some indicia to notify the clinician performing the procedure of the patient's state (step 708). Moreover, a reading outside of the prescribed ranges may require more extreme action, such as ending the procedure (step 710). In either case, illustrative embodiments may produce some visual (e.g., a flashing red light when outside the ranges, and a flashing yellow light when in the wider range) and/or audible indicia (e.g., an alarm).

To avoid the risk of certain short duration events (spikes) from affecting the procedure, various embodiments may require the out-of-range readings to have a consecutive duration of some specific time period. For example, the system may require the heart rate to be below 45 seconds for at least 5 seconds before taking action, or require the heart rate to be at 95 beats per minute for 30 seconds before taking action. Other embodiments may monitor for certain patterns (e.g., non-consecutive duration issues) indicating a problem.

The system preferably uses one of three techniques for converting the pressure values into the desired physiological value(s). Specifically, those three techniques include 1) Sine wave regression, 2) Fourier transform, and 3) Linear regression. To that end, FIGS. 8A, 9A, and 10A show three different processes that each use one of the noted techniques. These three processes have three similar steps.

Specifically, during the medical procedure, each process receives pressure signals from the lumbar and ventricular regions of the patient's body (steps 800, 900, and 1000). Accordingly, the pressure sensors noted above receive and forward pressure sensor information to the system with a prescribed frequency. The inventors discovered that higher frequency sampling rates of the pressure signal from the ventricle and/or lumbar provided improved results. For example, in some instances, sample rates of the incoming pressure signal of 30-120 Hertz are expected to produce satisfactory results. Other sampling rates also may provide satisfactory results, such as rates higher than 120 Hertz (e.g., 120-240 Hertz). Preferably, the sample rates of both the ventricle and lumbar pressure signals are the same, although some embodiments may have differing sample rates.

The inventors recognized that proper positioning of the lumbar and ventricular pressure sensors relative to the pump and the vessel is important for properly processing and comparing pressure signals from raw sensor output to actual pressure of target vessel. Distance to the vessel and geometry of the tubing line can be accounted for using standard fluid mechanics equations, such as the Hagen-Poiseuille Equation, which can correct the reported pressure (i.e., Δp=pressure difference between the two ends) based on the known constants of Volumetric Flow Rate (Q), tubing radius (R), length of tubing (L), and CSF viscosity (μ).

$$Δ p = \frac{8 μ LQ}{π {R}^{4}}$$

The next two steps of these processes of FIGS. 8A, 9A, and 10A apply their specific techniques to the received pressure signals and determine the parameters (discussed individually below). The final shown steps of these respective processes of FIGS. 8A, 9A, and 10A then stores the parameters, which were determined using the noted pressure signals from their first shown steps, in a patient database. This information may be used for later-performed procedures to optimize baseline information, ranges, etc. for future CSF fluid procedures (e.g., infusing a drug via the CSF at a later date). In some embodiments, artificial intelligence, neural networks, and/or machine learning techniques may use this and other information to further optimize future similar processes.

Each of the specific techniques of FIGS. 8A, 9A, and 10A now is discussed immediately below.

FIG. 8A, steps 802 and 804, convert, using Fourier transforms, the received pressure signals as shown in FIG. 8B and determine the desired physiological values by matching ventricular and lumbar pressure frequencies. Specifically, in FIG. 8B, the top graph shows the filtered ventricular pressure (purple curve starting around −1.0) and lumbar pressure (yellow curve starting around 0.5) over time. At the time this data was recorded, heart rate was roughly 1.6 Hz and respiratory rate was roughly 0.5 Hz. These signals are converted, preferably in real-time or near real-time, into the lower graph (their Fourier transforms) showing the power of the signals at different frequencies. FIG. 8B lists the top lumbar frequencies and the top ventricle frequencies resulting from the Fourier transform. As shown in this example, the lower graph shows two spikes—one at about 0.4 Hz (respiratory rate) and the other at about 1.6 Hz (heart rate), which represent the highest power frequencies for both the lumbar and ventricle data. These points may be points of high intersection of the basic pressure signals. Those frequency values then may be processed to determine the actual physiological parameter (e.g., simply multiplying the frequency by 60).

Specifically, a Fast Fourier Transform (aka “FFT”) may find common frequencies from the pressure data. In this example, as the data was recorded, the heart rate was about 1.6 Hertz while the respiratory rate was about 0.5 Hertz. The inventors recognized that physiologically relevant frequencies at the peak frequencies are shared by the lumbar and ventricle pressure data.

The inventors corroborated those observations with independent commercial instruments recording heart rate and respiratory rate (e.g., —Fast Fourier Transform reported 0.5 Hz, independent respiratory monitor reported 30 Breaths per min at the same time point). In practice, frequencies may be recorded and analyzed for matches between the two access points. Matches then are analyzed for clinical relevance by assessing the frequency against a database or user input (e.g., —Matching Frequency=0.4 (24 Breaths per min), average human resting respiratory rate range=20 to 30 breaths per minute, or physician inputs known patient specific respiratory rate range. Thus, the frequency can be analyzed and inferred as respiratory rate.

The inventors noticed that the pump sometimes affects the pressure data. As such, some embodiments use a filter to remove the undesired interference signal from the pump. FIG. 8C graphically shows one example of live physiologic data interference using Fourier Transform and band-pass filters. In that case, the left graph shows the raw data (i.e., the pressure data) from the patient. For example, a band-pass filter may isolate the pump signal (the center graph), while keeping the required information to determine the physiological value (e.g., respiration rate) as shown in the right graph. Other embodiments may use a high pass and/or low pass filter to further remove noise from the pressure signal. Still other embodiments may use other techniques to mitigate pump interference, e.g., processing pressure signals during intervals when the pump is inactive (e.g., alternating between pumping intervals and monitoring intervals) or when CSF pressures have settled or normalized during a pumping interval.

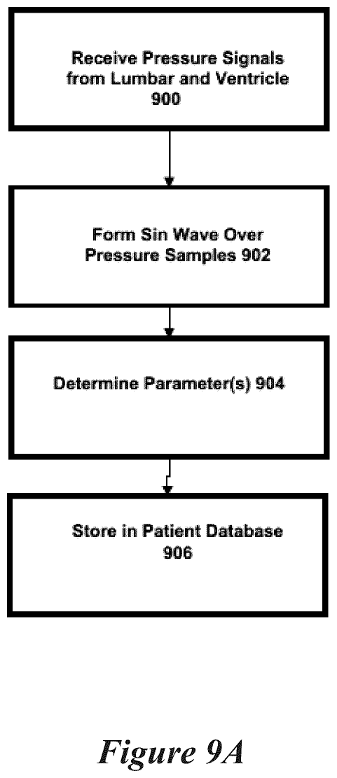

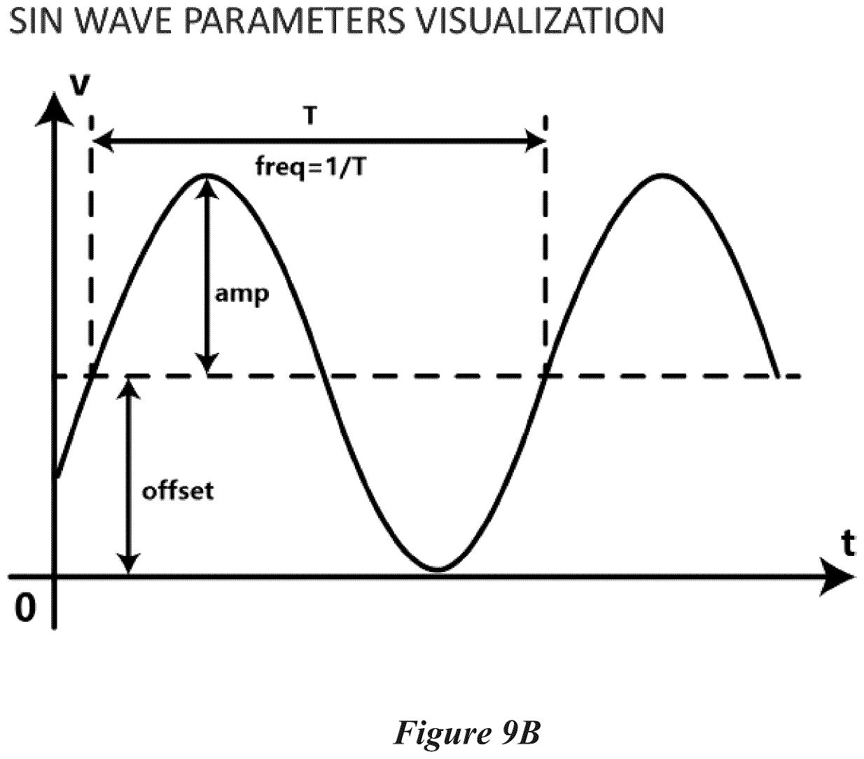

As a second example, steps 902 and 904 of FIG. 9A convert the input pressure data, using sine wave regression techniques, into the desired physiological values. Specifically, as known by those in the art and shown generically in FIG. 9B, this technique attempts a “best-fit” sine wave over raw data, when applicable, and outputs the error between the actual data and the sine wave. Data may be analyzed in time sections, such as ranging from 20 seconds to 20 minutes. If the error is below a certain threshold measured by a parameter, such as covariance, it may be analyzed for the parameters that compose a sine wave. As shown in FIG. 9B, parameters of the sine wave technique may include the offset, amplitude, phase, frequency, and period. These parameters then may be stored in memory.

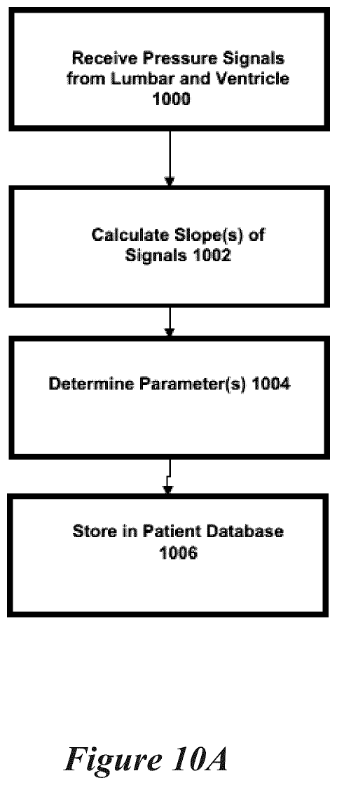

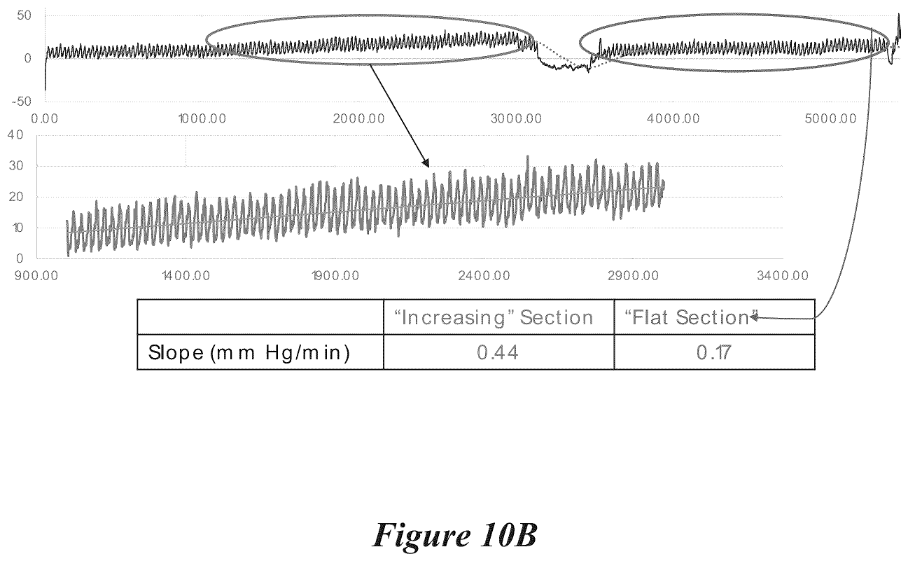

As a third example, steps 1002 and 1004 of FIG. 10A convert the input pressure data, using linear regression techniques, into the desired physiological values. Linear regression of the pressure versus time provides the slope of a “best-fit” line with units such as mmHg/min. Specifically, with FIG. 10B as an example of calculating CSF production rate using slope, when using this technique(s), a continuously positive slope can indicate the rate at which a parameter (e.g., fluid influx to the ventricle or access site) is greater than the difference between the CSF production and CSF reabsorption. This produces an increasing fluid volume and thus, increasing ventricular pressure typically observable over a longer length of time (e.g., 300 secs). Illustrative embodiments may convey, via a graphical user interface or similar device, that there is a constant positive slop for a specific time period (e.g., 300 seconds). The system may capture macro-data and assesses if there are seconds of the line of notable slope increases compared to the average slope (e.g., increasing vs. flat sections with a 2-3 times greater difference). Slope may typically be defined as pressure over time where:
1) “flat” sections of data may be characterized as having a slope close to 0.00+/−0.1 mmHg/min (i.e., no increase in pressure over time), or
2) “increasing” or “decreasing” sections where, for sections greater than 300 seconds, there is a 2 or greater multiplier of the “flat” section in either the negative or positive direction.

The three techniques noted above can be used separately or together to improve the fidelity and precision of the results. For example, some embodiments may use two or three of the techniques together, merge their results, and use the composite output data to make decisions or ascertain relevant physiological values (e.g., respiration or heart rates).

The inventors discovered that the following physiological values may be determine individually, or in combination, as shown the below table:

| # | CSF Variable | Process 1 | Process 2 | Parameter(s) |
|---|---|---|---|---|
| 1 | Intracranial Pressure | Sin Wave | Fourier | Offset |
| | | Optimization | Transform | (mmHg) |
| 2 | Lumbar Pressure | Sin Wave | Fourier | Offset |
| | | Optimization | Transform | (mmHg) |
| 3 | Heart Rate | Sin Wave | Fourier | Frequency |
| | | Optimization | Transform | (Hz) |
| 4 | Respiratory Rate | Sin Wave | Fourier | Frequency |
| | | Optimization | Transform | (Hz) |
| 5 | Heart Pulse Strength | Sin Wave | Fourier | Amplitude |
| | | Optimization | Transform | (mmHg) |
| 6 | Respiratory Pulse | Sin Wave | Fourier | Amplitude |
| | Strength | Optimization | Transform | (mmHg) |
| 7 | Vessel Compliance | Sin Wave | Linear | Phase Shift |
| | | Optimization | Regression | (Sec) |
| | | | | Frequency (Hz) |
| 8 | CSF Production Rate | Linear | N/A | Slope |
| | | Regression | | (mmHg/Sec) |
| 9 | Cerebral Perfusion | Sin Wave | Linear | Offset |
| | Pressure | Optimization | Regression | (mmHg) |
| 10 | Occlusion Prediction | Linear | N/A | Slope |
| | | Regression | | (mmHg/Sec) |

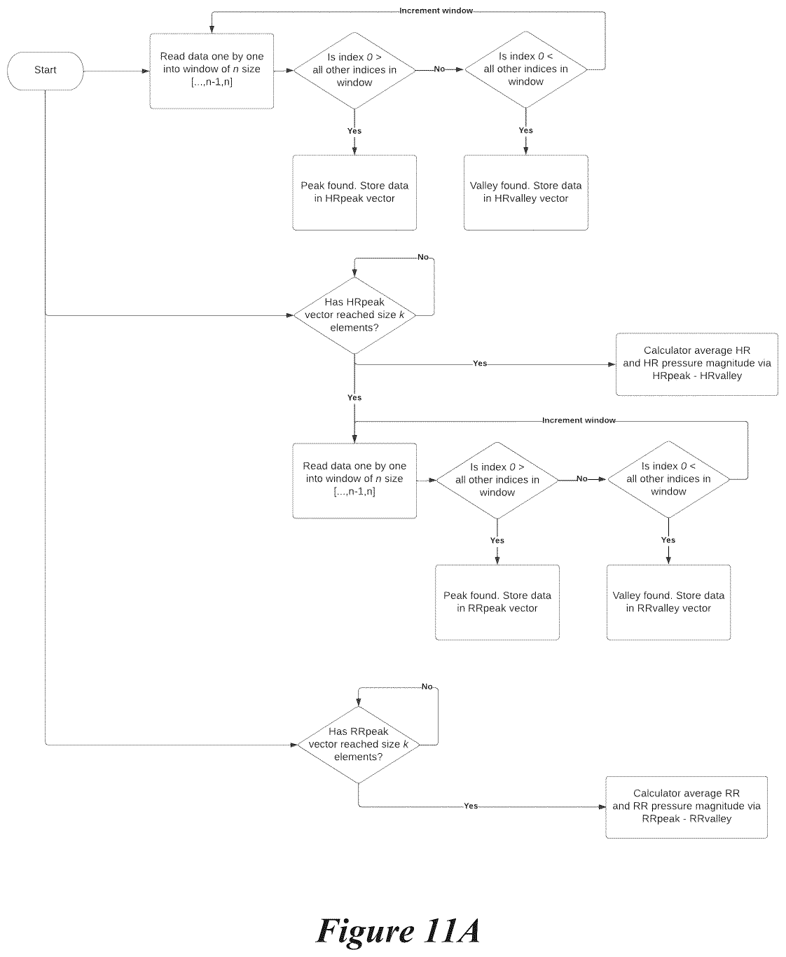

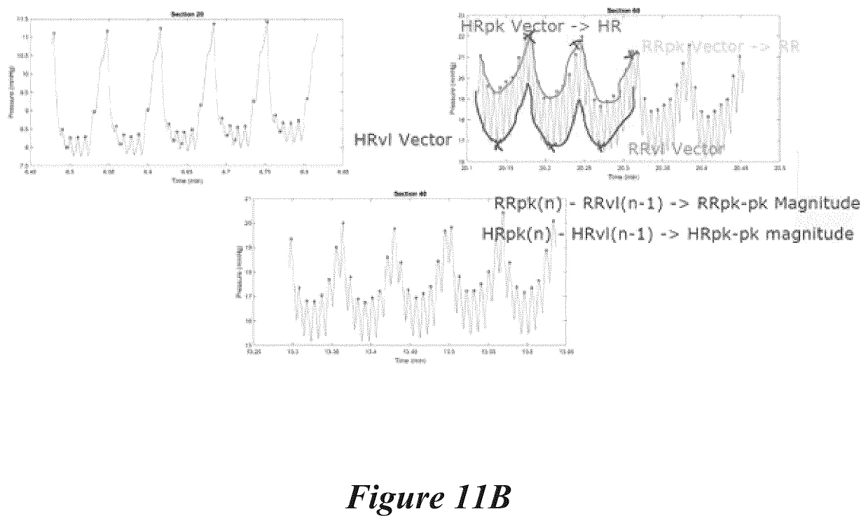

FIG. 11A shows a peak/valley process of determining heart and respiration parameters in accordance with illustrative embodiments. In this embodiment, rather than converting pressure readings into frequencies such as through Fourier transforms, the pressure readings are processed to identify local peaks and valleys to determine both heart rate and magnitude and respiration rate and magnitude. Specifically, pressure readings are examined within a sequence of windows with each window containing a predetermined number of pressure readings, e.g., on a sliding window basis or by examining discrete windows. If a particular pressure reading is the greatest within a window, then that pressure reading represents a local peak. If a particular pressure reading is the lowest within a window, then that pressure reading represents a local valley. The heart or respiration rate can be determined based on the distance in time between peaks. The heart or respiration magnitude can be determined based on the differential between peaks and valleys.

The process shown in FIG. 11A has two loops, a top loop for characterizing heart function and a bottom loop for characterizing respiration function. The input to the top loop is the “HRpeak” data vector, and the output of the top loop corresponds to the top purple curve shown in FIG. 11B, where the peaks marked with red asterisks represent the heartbeats, the distance between the peaks represent the heartbeat rate, and the differential between the peaks and valleys of that curve represent the heartbeat magnitude. The input to the bottom loop is the “RRpeak” data vector resulting from the top loop, and the output of the bottom loop corresponds to the bottom red curve shown in FIG. 11B, wherein the valleys marked with red asterisks represent the respirations, the distance between the valleys represents the respiration rate, and the differential between the peaks and valleys of that curve represents the respiration magnitude. Thus, even thought the top and bottom loops uses essentially the same logic, they operate on different data vectors and therefore produce different outputs, one relating to heart function and the other relating to respiration function.

It should be noted that impedance can be calculated using flow and pressure sensors. For example, a flow sensor can be placed in series with the pressure sensors. Signals from the flow sensor then can be compared with signals from the pressure sensors. A phase shift in the flow signal and the pressure signal can indicate the impedance or “stiffness” of the vessel into which the fluid is flowing. This stiffness can indicate how CSF will flow through the target vessel, and could indicate drug delivery to structures downstream. This impedance parameter also could be stored, e.g., as a comparator for optimal drug delivery.

It also should be noted that pressure signals from the ventricle pressure sensor, the lumbar pressure sensor, and/or other pressure sensor can be used to determine if a catheter is properly placed in the ventricle, lumbar, or other CSF access location (e.g., cisterna magna). The process of locating the site of injection such as the lateral ventricle in the brain is not simple, and generally involves advanced imaging systems such as Fluoroscopy, MRI, and real time imaging devices such as Brain-Sight. Even with these systems in place, it is still difficult to understand if the needle is correctly in the vessel, and if it is patent. When properly placed, it is expected that the CSF (and therefore the CSF pressure signals) will vary with the heart rate, and therefore pressure signals analyzed by the system or displayed on a graphical user interface can be used to determine if the catheter is properly placed. If the pressure waveform shows the typical shape of physiologic signals, the surgical team can be confident that the vessel has been accessed and that it is patent. Conversely, if the pressure wave form is flat, or inconclusive, the surgical team can adjust the needle position (such as lowering the needle deeper or shallower or rotating the needle) until the waveform displays the physiologic signal.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-along hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:
P1. A method of determining respiration rate or heart rate, the method comprising:
fluidly communicating a catheter with the patient's brain ventricle;
fluidly communicating the catheter with the patient's lumbar;
detecting, using a ventricle pressure sensor in fluid communication with the catheter, the brain ventricle pressure to produce a ventricle pressure signal;
detecting, using a lumbar pressure sensor in fluid communication with the catheter, the lumbar pressure to produce a lumbar pressure signal;
using the ventricle pressure signal and the lumbar pressure signal to determine respiration rate and/or heart rate.
P2. The method of claim P1 wherein using comprises applying a Fourier Transform to the lumbar and ventricle pressure signals.
P3. The method of one or more of the above potential claims wherein using comprises forming a Fourier Transform from the lumbar and ventricle pressure signals and locating at least one peak from the Fourier Transform, the at least one peak corresponding with at least one of the respiration rate and the heart rate.
P4. The method of one or more of the above potential claims wherein using comprises locating a point of intersection between the lumbar and ventricle pressure signals, the point of intersection corresponding with at least one of the heart rate and the respiration rate.
P5. The method of one or more of the above potential claims wherein using comprises applying a sin wave to one or both to the lumbar and ventricle pressure signals, using further comprising ascertaining one or more physiological parameters based on one or more of the amplitude, offset, frequency, slope, and period of the sin wave, the one or more physiological parameters comprising at least one of the heart rate, respiration rate, cerebral perfusion pressure, intracranial pressure, heart rate amplitude, respiration rate amplitude, vessel elasticity/impedance, cerebral compliance, and CSF production rate.
P6. The method of one or more of the above potential claims wherein using comprises determining the slope of the lumbar and/or ventricle pressure signals, the heart rate and/or the respiration rate being a function of the slope.
P7. The method of one or more of the above potential claims further comprising monitoring the determined heart rate and/or the respiration rate and producing indicia when the heart rate and/or the respiration rate extend beyond a prescribed range relative to a baseline reading, the indicia being visual, audio, and/or tactile indicia.
P8. The method of one or more of the above potential claims further comprising taking a baseline reading using the lumbar and/or ventricle pressure signals to determine heart rate and/or respiration rate, the method further comprising storing the baseline reading in a database.
P9. The method of one or more of the above potential claims wherein the catheter has a ventricle end coupled with the ventricle, the catheter having a lumbar end coupled with the lumbar, the lumbar pressure sensor being closer to the lumbar end than the ventricle end, and the ventricle pressure sensor being closer to the ventricle end than the lumbar end.
P10. The method of one or more of the above potential claims wherein using comprises applying two or more of a Fourier Transform, sin wave, and slope techniques to the ventricle and lumbar pressure signals and using the result of that application to determine the respiration rate and/or the heart rate.
P11. A computer program product for use on a computer system for determining a respiration rate and/or a heart rate, the computer program product comprising a tangible, non-transient computer usable medium having computer readable program code thereon, the computer readable program code comprising instructions configured in accordance with any one or more of claims P1-P10.
P12. A CSF system comprising:
a catheter having a lumbar end and a ventricle end;
a lumbar pressure sensor in fluid communication with the lumbar end, the lumbar pressure sensor being closer to the lumbar end than to the ventricle end, the lumbar pressure sensor configured to produce a lumbar pressure signal;
a ventricle pressure sensor in fluid communication with the lumbar end, the ventricle pressure sensor being closer to the ventricle end than to the lumbar end, the ventricle pressure sensor configured to produce a ventricle pressure signal;
a physiological parameter monitor operatively coupled with the ventricle pressure sensor and the lumbar pressure sensor, the physiological parameter monitor configured to use the ventricle and lumbar pressure signals to determine the respiration rate and/or the heart rate.
P13. The system of claim P12 wherein the physiological parameter monitor is configured to apply a Fourier Transform to the lumbar and ventricle pressure signals.
P14. The system of one or more of the above claims P12-P13 wherein the physiological parameter monitor is configured to form a Fourier Transform from the lumbar and ventricle pressure signals and locating at least one peak from the Fourier Transform, the at least one peak corresponding with at least one of the respiration rate and the heart rate.
P15. The system of one or more of claims P12-P14 wherein the physiological parameter monitor is configured to locate a point of intersection between the lumbar and ventricle pressure signals, the point of intersection corresponding with at least one of the heart rate and the respiration rate.
P16. The system of one or more of claims P12-P15 wherein the physiological parameter monitor is configured to apply a sin wave to one or both to the lumbar and ventricle pressure signals, using further comprising ascertaining one or more physiological parameters based on one or more of the amplitude, offset, frequency, slope, and period of the sin wave, the one or more physiological parameters comprising at least one of the heart rate, respiration rate, cerebral perfusion pressure, intracranial pressure,
heart rate amplitude, respiration rate amplitude, vessel elasticity/impedance, cerebral compliance, and CSF production rate.
P17. The system of one or more of claims P12-P16 wherein the physiological parameter monitor is configured to determine the slope of the lumbar and/or ventricle pressure signals, the heart rate and/or the respiration rate being a function of the slope.
P18. The system of one or more of claims P12-P17 wherein the physiological parameter monitor is configured to apply two or more of a Fourier Transform, sin wave, and slope techniques to the ventricle and lumbar pressure signals and using the result of that application to determine the respiration rate and/or the heart rate.
P19. The system of one or more of claims P12-P18 wherein the physiological parameter monitor is configured to monitor the heart rate and/or the respiration rate and cause production of indicia when the heart rate and/or the respiration rate extend beyond a prescribed range relative to a baseline reading, the indicia being visual, audio, and/or tactile indicia.
P20. The system of one or more of claims P12-P19 further comprising a database for storing a baseline reading using the lumbar and/or ventricle pressure signals to determine heart rate and/or respiration rate.

The embodiments of the invention described above are intended to be merely exemplary; numerous variations and modifications will be apparent to those skilled in the art. Such variations and modifications are intended to be within the scope of the present invention as defined by any of the appended claims.

1. A method for monitoring cardiopulmonary function of a patient based on cerebrospinal fluid (CSF) pressures, the method comprising:
fluidly communicating a first catheter with a first CSF access site of the patient;
fluidly communicating a second catheter with a second CSF access site of the patient;
providing, using a first pressure sensor in fluid communication with the first catheter, first pressure readings associated with the first CSF access site;
providing, using a second pressure sensor in fluid communication with the second catheter, second pressure readings associated with the second CSF access site; and
determining a cardiopulmonary function parameter associated with the patient based on first and second pressure signals derived from the first and second pressure readings.

2. The method of claim 1, wherein the first CSF access site is associated with a brain ventricle of the patient and the second CSF access site is associated with the patient's lumbar.

3. (canceled)

4. (canceled)

5. (canceled)

6. (canceled)

7. (canceled)

8. (canceled)

9. The method of claim 1, further comprising taking a baseline reading using the first and/or second pressure signals to determine a baseline for the cardiopulmonary function parameter, the method further comprising storing the baseline reading in a database.

10. The method of claim 1, wherein a catheter has a first end coupled with the first CSF access site and a second end coupled with the second CSF access site, the first pressure sensor being closer to the first end than the second end, and the second pressure sensor being closer to the second end than the first end.

11. (canceled)

12. The method of claim 1, wherein the cardiopulmonary function parameter comprises at least one of heart rate, respiration rate, heart rate amplitude, respiration rate amplitude, or vessel compliance.

13. The method of claim 1, further comprising:
fluidly communicating a third catheter with a third CSF access site of the patient;
detecting, using a third pressure sensor in fluid communication with the third catheter, third pressures and producing third pressure signals based on the third pressures; and
using the third pressure signals in combination with the first pressure signals and the second pressure signals to determine the cardiopulmonary function parameter.

14. (canceled)

15. (canceled)

16. A system for monitoring cardiopulmonary function of a patient based on cerebrospinal fluid (CSF) pressures, the system comprising:
a first catheter for fluidly communicating with a first CSF access site of the patient;
a second catheter for fluidly communicating with a second CSF access site of the patient;
a first pressure sensor in fluid communication with the first catheter to provide first pressure readings associated with the first CSF access site;
a second pressure sensor in fluid communication with the second catheter to provide second pressure readings associated with the second CSF access site; and
a physiological parameter monitor operatively coupled to receive the first and second pressure readings from the first and second pressure sensors and to determine a cardiopulmonary function parameter associated with the patient based on first and second pressure signals derived from the first and second pressure readings.

17. The system of claim 16, wherein the first CSF access site is associated with a brain ventricle of the patient and the second CSF access site is associated with the patient's lumbar.

18. (canceled)

19. (canceled)

20. (canceled)

21. (canceled)

22. (canceled)

23. (canceled)

24. The system of claim 16, wherein the physiological parameter monitor is configured to take a baseline reading using the first and/or second pressure signals to determine a baseline for the cardiopulmonary function parameter, and wherein the physiological parameter monitor is configured to store the baseline reading in a database.

25. The system of claim 16, wherein a catheter has a first end coupled with the first CSF access site and a second end coupled with the second CSF access site, the first pressure sensor being closer to the first end than the second end, and the second pressure sensor being closer to the second end than the first end.

26. (canceled)

27. The system of claim 16, wherein the cardiopulmonary function parameter comprises at least one of heart rate, respiration rate, heart rate amplitude, respiration rate amplitude, or vessel compliance.

28. The system of claim 16, further comprising:
a third catheter for fluidly communicating with a third CSF access site of the patient; and
a third pressure sensor in fluid communication with the third catheter to provide third pressure readings associated with the first CSF access site, wherein the physiological parameter monitor is further configured to receive the third pressure readings from the third pressure sensor and to determine the cardiopulmonary function parameter associated with the patient based on third pressure signals derived from the third pressure readings in combination with the first and second pressure signals.

29. (canceled)

30. (canceled)

31. A computer program product comprising at least one tangible, non-transitory computer-readable medium having embodied therein computer program instructions for monitoring cardiopulmonary function of a patient based on cerebrospinal fluid (CSF) pressures, which, when executed by at least one processor of a computer system, causes the computer system to perform processes comprising:
receiving, from a first pressure sensor in fluid communication with a first catheter, first pressure readings associated with a first CSF access site;
receiving, from a second pressure sensor in fluid communication with a second catheter, second pressure readings associated with a second CSF access site; and
determining a cardiopulmonary function parameter associated with the patient based on first and second pressure signals derived from the first and second pressure readings.

32. The computer program product of claim 31, wherein the first CSF access site is associated with a brain ventricle of the patient and the second CSF access site is associated with the patient's lumbar.

33. (canceled)

34. (canceled)

35. (canceled)

36. (canceled)

37. (canceled)

38. (canceled)

39. The computer program product of claim 31, wherein the processes further comprise taking a baseline reading using the first and/or second pressure signals to determine a baseline for the cardiopulmonary function parameter, and storing the baseline reading in a database.

40. The computer program product of claim 31, wherein a catheter has a first end coupled with the first CSF access site and a second end coupled with the second CSF access site, the first pressure sensor being closer to the first end than the second end, and the second pressure sensor being closer to the second end than the first end.

41. (canceled)

42. The computer program product of claim 31, wherein the cardiopulmonary function parameter comprises at least one of heart rate, respiration rate, heart rate amplitude, respiration rate amplitude, or vessel compliance.

43. The computer program product of claim 31, wherein the processes further comprise receiving, from a third pressure sensor in fluid communication with a third catheter, third pressure readings associated with a third CSF access site, wherein determining comprises determining the cardiopulmonary function parameter associated with the patient based on third pressure signals derived from the third pressure readings in combination with the first and second pressure signals.

44. (canceled)

45. (canceled)

46. The method of claim 1, wherein determining comprises at least one of:
applying a Fourier Transform to the first and second pressure signals;
forming a first Fourier Transform from the first pressure signals, forming a second Fourier Transform from the second pressure signals, and locating at least one peak from the first and second Fourier Transforms, the at least one peak corresponding with the cardiopulmonary function parameter;
locating a point of intersection between the first and second pressure signals, the point of intersection corresponding with the cardiopulmonary function parameter;
applying a sine wave to one or both of the first and second pressure signals, using further comprising ascertaining the cardiopulmonary function parameter based on one or more of the amplitude, offset, frequency, slope, and period of the sine wave;
determining the slope of the first and/or second pressure signals, the cardiopulmonary function parameter being a function of the slope;
applying two or more of a Fourier Transform, sine wave, or slope techniques to the first and second pressure signals and using the result of that application to determine the cardiopulmonary function parameter;
characterizing a first pressure contribution associated with the first catheter, producing the first pressure signals based on the first pressure readings and the first pressure contribution, characterizing a second pressure contribution associated with the second catheter, and producing the second pressure signals based on the second pressure readings and the second pressure contribution; or
applying a peak/valley process to the first and second pressure signals to determine the cardiopulmonary function parameter.

47. The system of claim 16, wherein the physiological parameter monitor is configured to perform at least one of:
applying a Fourier Transform to the first and second pressure signals;
forming a first Fourier Transform from the first pressure signals, forming a second Fourier Transform from the second pressure signals, and locating at least one peak from the first and second Fourier Transforms, the at least one peak corresponding with the cardiopulmonary function parameter;
locating a point of intersection between the first and second pressure signals, the point of intersection corresponding with the cardiopulmonary function parameter;
applying a sine wave to one or both of the first and second pressure signals, using further comprising ascertaining the cardiopulmonary function parameter based on one or more of the amplitude, offset, frequency, slope, and period of the sine wave;
determining the slope of the first and/or second pressure signals, the cardiopulmonary function parameter being a function of the slope;
applying two or more of a Fourier Transform, sine wave, or slope techniques to the first and second pressure signals and using the result of that application to determine the cardiopulmonary function parameter;
characterizing a first pressure contribution associated with the first catheter, producing the first pressure signals based on the first pressure readings and the first pressure contribution, characterizing a second pressure contribution associated with the second catheter, and producing the second pressure signals based on the second pressure readings and the second pressure contribution; or
applying a peak/valley process to the first and second pressure signals to determine the cardiopulmonary function parameter.

48. The computer program product of claim 31, wherein determining comprises at least one of:
applying a Fourier Transform to the first and second pressure signals;
forming a first Fourier Transform from the first pressure signals, forming a second Fourier Transform from the second pressure signals, and locating at least one peak from the first and second Fourier Transforms, the at least one peak corresponding with the cardiopulmonary function parameter;
locating a point of intersection between the first and second pressure signals, the point of intersection corresponding with the cardiopulmonary function parameter;
applying a sine wave to one or both of the first and second pressure signals, using further comprising ascertaining the cardiopulmonary function parameter based on one or more of the amplitude, offset, frequency, slope, and period of the sine wave;
determining the slope of the first and/or second pressure signals, the cardiopulmonary function parameter being a function of the slope;
applying two or more of a Fourier Transform, sine wave, or slope techniques to the first and second pressure signals and using the result of that application to determine the cardiopulmonary function parameter;
characterizing a first pressure contribution associated with the first catheter, producing the first pressure signals based on the first pressure readings and the first pressure contribution, characterizing a second pressure contribution associated with the second catheter, and producing the second pressure signals based on the second pressure readings and the second pressure contribution; or
applying a peak/valley process to the first and second pressure signals to determine the cardiopulmonary function parameter.
